(19) United States
(12) Patent Application Publication
(10) Pub. No.: US 2024/0385167 A1
(43) Pub. Date: Nov. 21, 2024

(54) APPARATUS AND METHOD TO EVALUATE THE EFFECT OF ELECTRICAL POTENTIAL BETWEEN MOVING SURFACES

(71) Applicants: Southwest Research Institute, San Antonio, TX (US); PCS Instruments Ltd, London (GB)

(72) Inventors: Carlos Joel SANCHEZ, San Antonio, TX (US); Peter Mark LEE, Fair Oaks Ranch, TX (US); John Clive HAMER, London (GB); Thomas William George WELHAM, Maidenhead (GB); Benjamin Philip WAINWRIGHT, London (GB); Matthew SMEETH, London (GB)

(21) Appl. No.: 18/665,830

(22) Filed: May 16, 2024

Related U.S. Application Data
(60) Provisional application No. 63/502,515, filed on May 16, 2023.

(51) Int. Cl. G01N 33/28; G01K 7/02; G01N 19/02

(52) U.S. Cl. CPC G01N 33/2888; G01K 7/02; G01N 19/02

(57) ABSTRACT

An apparatus and method to evaluate the effect of electrical potential between moving surfaces and lubricants and/or oils under mechanical load.

### FIELD

The present invention is directed at an apparatus and method to evaluate the effect of electrical potential between moving surfaces and lubricants and/or oils under mechanical load.

### BACKGROUND

The influence of applied electrical potential on rubbing contacts, a topic sometimes identified as “tribo-electrochemistry” has assumed new attention with the development of hybrid electric and electric vehicles. Namely, an evaluation of lubricant performance on the components of electric-motor and gear box performance in light of exposure to electrical potentials. This is particularly of growing importance considering the observation that the impact of an applied electrical potential on friction and wear, in a lubricated system, may be beneficial or harmful, depending upon the nature of the applied potential and the characteristics of the frictionally engaged surfaces, as well as the selected lubricants employed for sliding and rolling type contact.

### SUMMARY

An electrically insulated ball on disk test unit comprising an electrically isolated rotating disk, an electrically isolated rotating ball configured to be mechanically loaded against the electrically isolated rotating disk, a negative electrode in electrical communication with the rotating ball and a positive electrode in electrical communication with the rotating disk, or a positive electrode in electrical communication with said ball and a negative electrode in communication with said disk, wherein the negative and positive electrodes are configured to supply an electrical potential between the electrically isolated rotating ball and the electrically isolated rotating disk.

A method for evaluating the effects of electrical potential on one or more lubricants or oils in a test unit comprising supplying a test unit having an electrically isolated rotating disk, an electrically isolated rotating ball configured to be mechanically loaded against the electrically isolated rotating disk, a negative electrode in electrical communication with the rotating ball and a positive electrode in electrical communication with the rotating disk, or vice-versa, wherein the negative and positive electrodes are configured to supply an electrical potential between the electrically isolated rotating ball and the electrically isolated rotating disk. This is followed by introducing one or more lubricants or oils between the electrically isolated rotating ball and the electrically isolated rotating disk. One may then rotate the ball and disk at different speeds and apply a mechanical load and electrical potential between the electrically isolated rotating ball and the electrically isolated rotating disk. The rotation of the ball and disk is preferably initiated before application of the mechanical load and electrical potential between the electrically isolated rotation ball and electrically isolated rotating disk.

### BRIEF DESCRIPTION OF THE DRAWINGS

FIG. 1 is a perspective view of the preferred test device of the present invention.

FIG. 2 provides a view of the circuity for the preferred test device of the present invention.

FIG. 3 is a graph of traction coefficients against speed on a log/log scale for automatic gear oil (AGO) on the electrically insulated ball on disk test unit herein at 100° C. with no potential and the indicated potentials.

FIG. 4 is a graph of showing a measure of wear track-volumetric wear for the same conditions in FIG. 3.

FIG. 5 is a traction curve for triplicate runs at 100° C. for ultralow viscosity automatic transmission fluid with no electrification as well as DC and AC.

FIG. 6 is a traction curve for triplicate runs at 100° C. for Dexron VI automatic transmission fluid with no electrification as well as DC and AC.

FIG. 7 is a traction curve for triplicate runs at 100° C. for automatic gear oil with no electrification as well as DC and AC.

FIG. 8 illustrate end of test volumetric wear for triplicate runs at 100° C. for the three (3) identified oils with no electrification, DC and AC.

### DETAILED DESCRIPTION

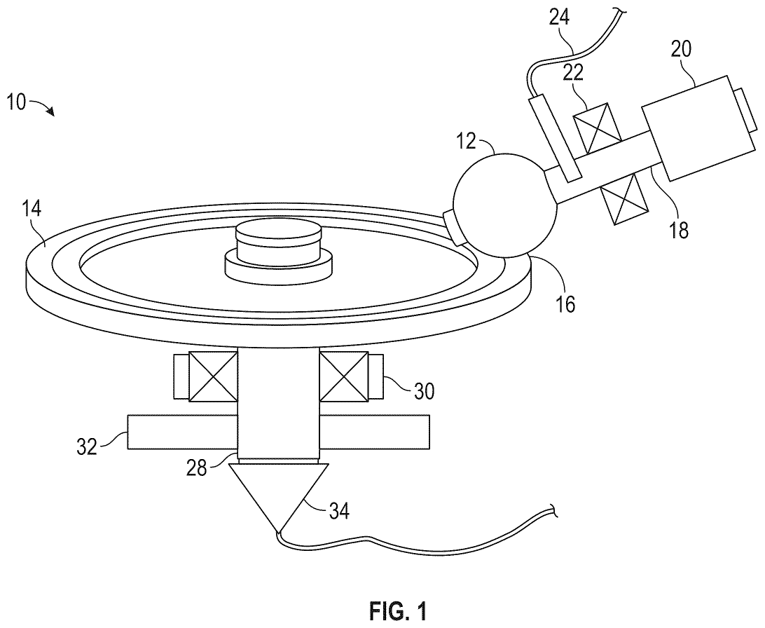

The present invention is directed at a test unit and method to investigate the effects of electrical potential of material pair surfaces in sliding and rolling contact and the lubricants or oils used therein. A view of a preferred testing device 10 is illustrated in FIG. 1. A lubricant is understood herein as a substance that assists to reduce friction between surfaces in mutual contact. An oil is reference to a liquid, typically derived from petroleum, that also serves as a lubricant.

As illustrated, test unit 10 includes an electrically isolated metallic or electrically conductive rotating metallic ball 12 and a rotating metallic or electrically conductive disk 14. Reference to electrically isolated means that the rotating ball 12 and the rotating disk 14 are not in electrical communication with any other components of the testing device 10. The rotating ball 12 and rotating disk 14 are configured such that a mechanical load can be applied between such components. An oil or lubricant may then be disposed at location 16 between the rotating ball 12 and rotating disk 14.

Preferably, the electrically conductive rotating ball 12 includes an electrically conductive shaft portion 18 which is engaged to a motor 20. The shaft portion is preferably supported by bearings 22 which are preferably formed of ceramic material which bearings preferably include non-conductive sleeves. An electrode is shown at 24 engaged to the shaft portion 18 to supply an AC or DC potential. The electrode is preferably a carbon-brush type electrode.

The rotating disk 14 includes an electrically conductive shaft portion 28. The rotating disk 14 is preferably supported by bearings 30 which are preferably formed of ceramic material and preferably include non-conductive sleeves. A mechanical drive is shown generally at 32. The electrically conductive shaft portion is then preferably coupled at 34, such as a mercury coupling, to allow for either positive or negative electrical supply.

The disk preferably rotates within a container containing the test lubricant or oil with the disk submerged in the lubricant. One or a plurality of external container heaters are configured to heat the lubricant or oil to a selected test temperature. The temperature may then be monitored using a thermocouple within the container and submerged in the lubricant.

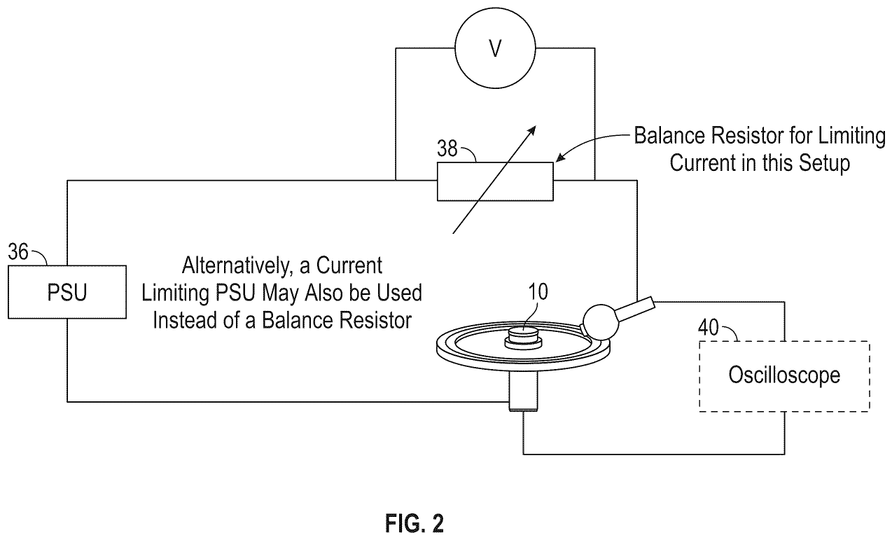

FIG. 2 provides a view of the circuity that may then be applied to testing device 10. As can be seen, a power supply unit is identified at 36 that may be current limiting. FIG. 2 also shows that in lieu of a current limiting power supply, one may employ a balance resistor 38 for limiting current provided to the test device 10.

An oscilloscope 40, or any other voltage measurement or visualization device, such as anything containing an analog to digital converter, or analog amplifier, may then be connected to the test device to monitor to provide an evaluation of the electrical properties of the oils or lubricants under an applied mechanical loading condition. That may include, but not be limited to an evaluation of the response of any selected oil or lubricant under a given electrical potential or varying electrical potential to the shearing force on the oil or lubricant applied by the mechanical load between the electrically conductive rotating metallic ball 12 and a rotating metallic or electrically conductive disk 14. By way of example, one may evaluate whether or not the applied electrical potential has any influence on the chemical composition of the oil or lubricant, and its ensuing lubricating properties, under a selected mechanical load condition.

Accordingly, one may evaluate herein the influence of oil or lubricant shearing, in the presence of an applied electrical potential (AC or DC) on the lubricating properties of a selected oil or lubricant, between a given material surface of the rotating metallic or electrically conductive ball 12 and rotating metallic or electrically conductive disk 14. DC is reference to direct current and AC is reference to alternating current of an arbitrary wave form. Polarities can be any or any combination of the following: (1) ball positive, disk negative; (2) disk positive, ball negative; (3) alternating current; (4) direct or alternating current with either the ball or disk grounded; (4) alternating current with DC offset applied to either the ball or disk; (5) both ball and disk with a positive or negative DC potential between them, both offset from ground, either positive or negative.

It is further contemplated that one may now also evaluate the impact of oil or lubricant aging, for a given applied potential, under selected shearing (disk vs. ball rpm) conditions for a given applied load on the metallic or electrically conductive ball 12 over a selected period of time. One may also evaluate differences in friction/traction under applied voltage/current as well differences in wear rates and arcing across the lubricant film.

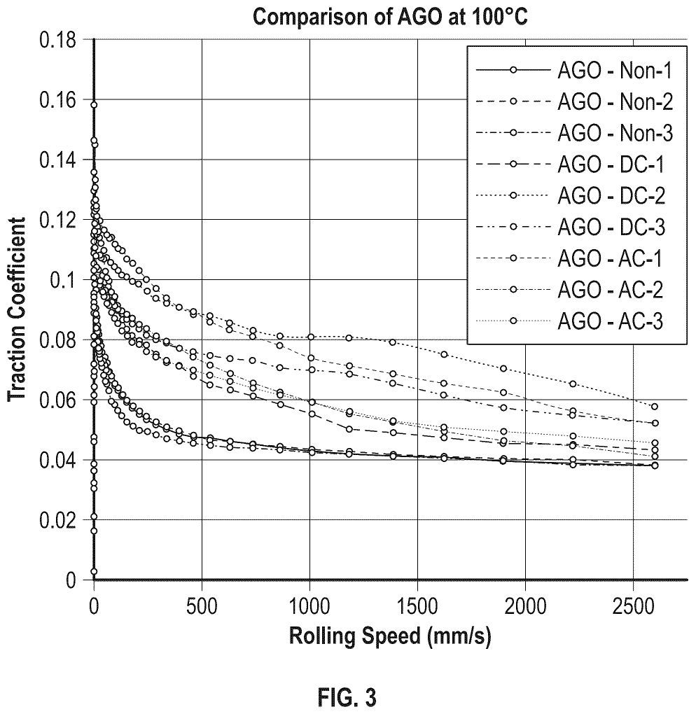

Attention is next directed to FIG. 3 which relies on the device herein showing traction coefficients against rolling speed on a log/log scale. This shows the Stribeck curve for automatic gear oil (AGO) at 100° C. A Stribeck curve illustrates the friction response versus speed. Two observations. First, when no electric potential is placed across the contact between the ball 12 and disk 14 (see FIG. 1), the traction coefficient is lowest and generally repeatable (see runs AGO Non-1, AGO Non-2 and AGO Non-3). When AC or DC is placed across the contact between the ball 12 and disk 14, the traction coefficient increases and becomes generally non-repeatable. In these runs, the AC current was 20 amps and the DC current was 3 amps.

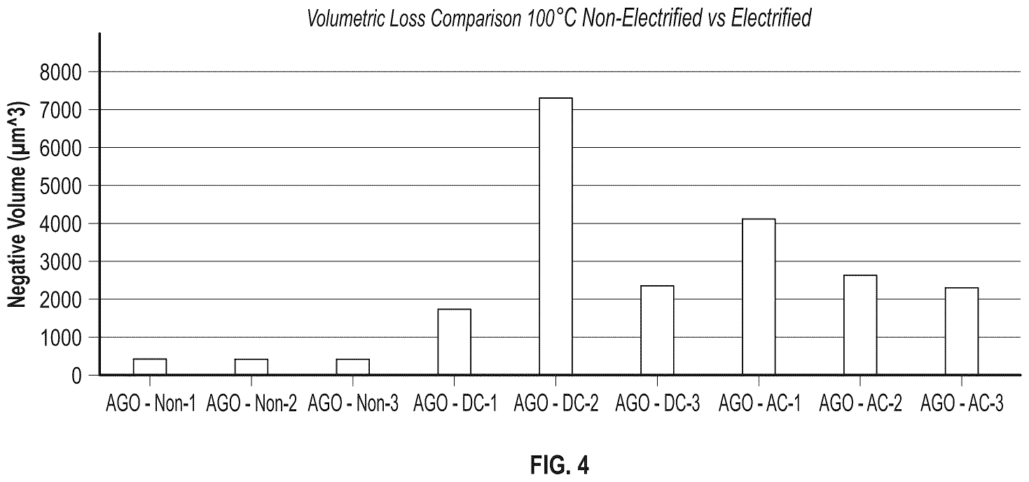

Attention is next directed to FIG. 4 which is a measure of the wear track-volumetric wear for the same conditions noted above. This graph shows that when end of test wear is measured after testing with no electrical contact between the ball 12 and disk 14 (see again FIG. 1), there is relatively low and repeatable wear. However, when AC or DC is applied, the wear increases.

As those of skill in the art will appreciate, all of the above-mentioned contemplated output metrics of device 10 will be of importance as the electrical fluid properties of oils and lubricants become increasingly significant with the growth of electrification in power-providing engine designs and configurations.

Additional testing proceeded as follows. Three different oils were employed for further wear and friction evaluation. The test lubricants were the following three specimens: MERCON® ULV, DEXRON®-VI, and OMALA J2360 gear oil. The MERCON® ULV and DEXRON®-VI are common oils used in automatic transmissions and are of a lower viscosity than the OMALA, which is a common oil used in differential applications (gearing that allows different drive wheels on the same axle to rotate at different speeds). Separate AC and DC power supplies were used to control different voltage configurations.

To maintain consistency between tests, balls and disks were purchased from PCS Instruments. Both the balls and disks are AISI 52100 super finished. Before testing, the samples were sonicated in hexane for 20 minutes at an elevated temperature to remove any debris. The samples were then weighed on a GR-300 scale. After testing, the samples followed the same rinsing and weighing process as performed during initial preparation to find mass loss/gain after testing. All tests were run in triplicate and the test procedure was a half hour run-in with no electrification followed by Stribeck curve measurements at the required test specification (i.e load, temperature and electrification). The full test matrix is shown below:

| Electrification | Temperature (C.) | Load (N) |
|---|---|---|
| None | 20 | 5 |
| | | 40 |
| | | 75 |
| | 40 | 5 |
| | | 40 |
| | | 75 |
| | 80 | 5 |
| | | 40 |
| | | 75 |
| | 100 | 5 |
| | | 40 |
| | | 75 |
| | 120 | 5 |
| | | 40 |
| | | 75 |
| DC | 20 | 5 |
| | | 40 |
| | | 75 |
| | 40 | 5 |
| | | 40 |
| | | 75 |
| | 80 | 5 |
| | | 40 |
| | | 75 |
| | 100 | 5 |
| | | 40 |
| | | 75 |
| | 120 | 5 |
| | | 40 |
| | | 75 |
| AC | 20 | 5 |
| | | 40 |
| | | 75 |
| | 40 | 5 |
| | | 40 |
| | | 75 |
| | 80 | 5 |
| | | 40 |
| | | 75 |
| | 100 | 5 |
| | | 40 |
| | | 75 |
| | 120 | 5 |
| | | 40 |
| | | 75 |

In total 145 tests were conducted. It was recognized that preferably, the electrification of the subject electrically insulated ball on disk test unit is turned on after the parts were operating (rotating of the ball relative to the disk). Likewise the electrification is preferably turned off before the test is stopped (no additional rotation of the ball relative to the disk). While the parts are operating, lubricant is entrained into the contact between the ball and the disk. When the parts are stationary, there is relatively minimal lubricant between them. If electrification is applied prior to the parts operating then as they separated an arc occurs between them, causing damage to the surface of the disk and the ball. Thus, the aforementioned preferred operation protocol.

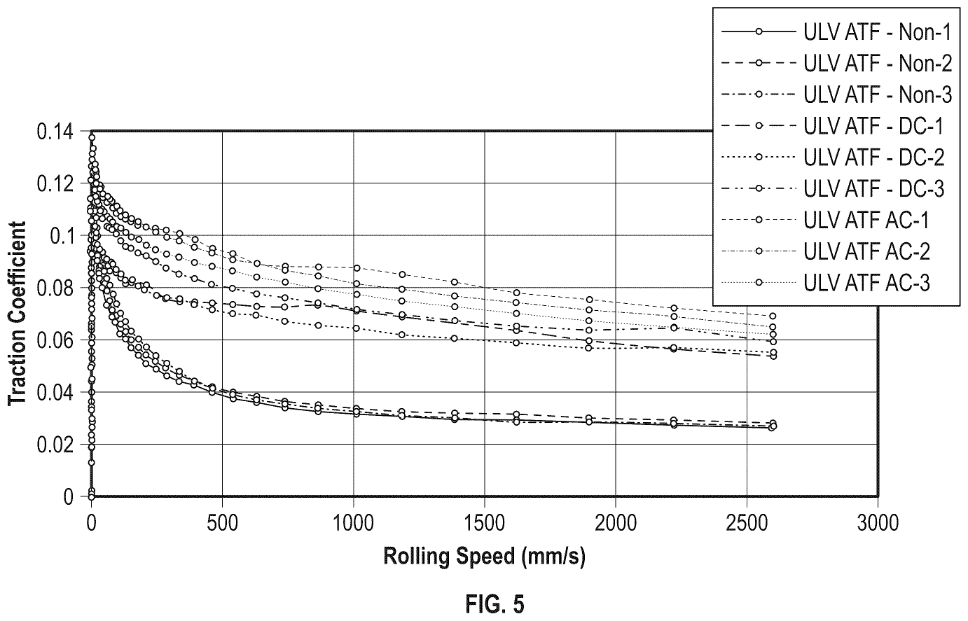

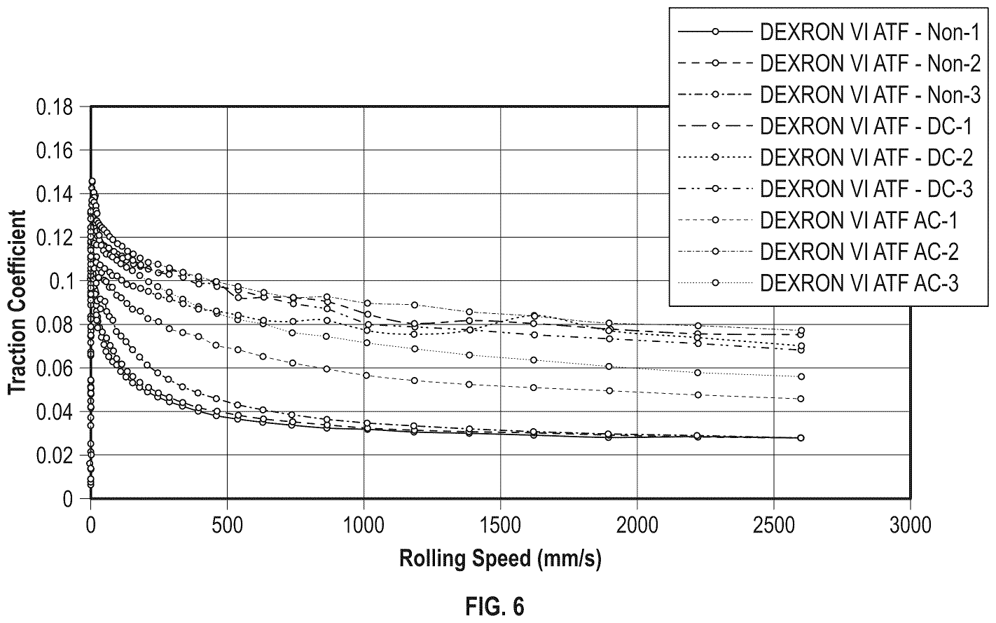

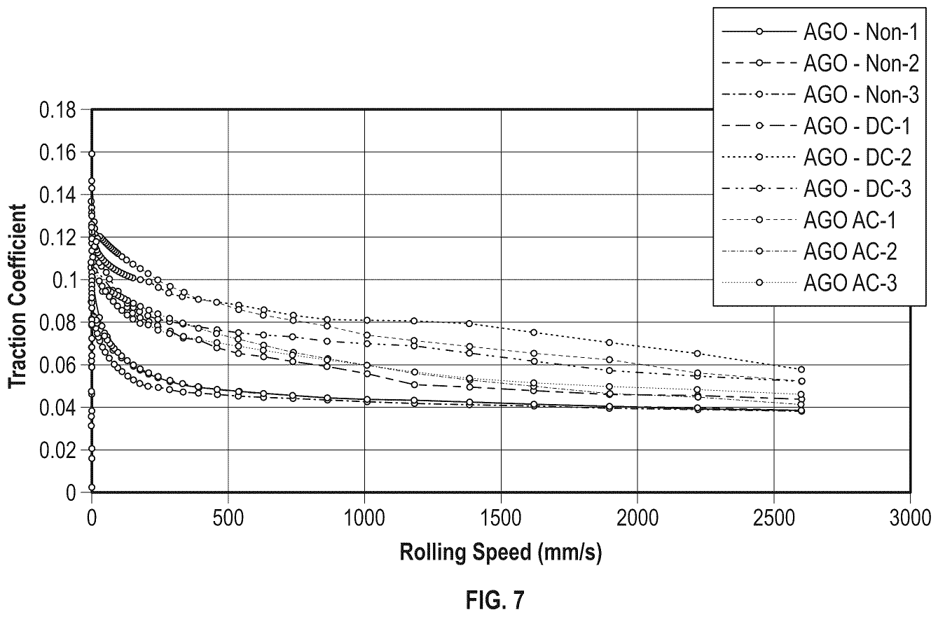

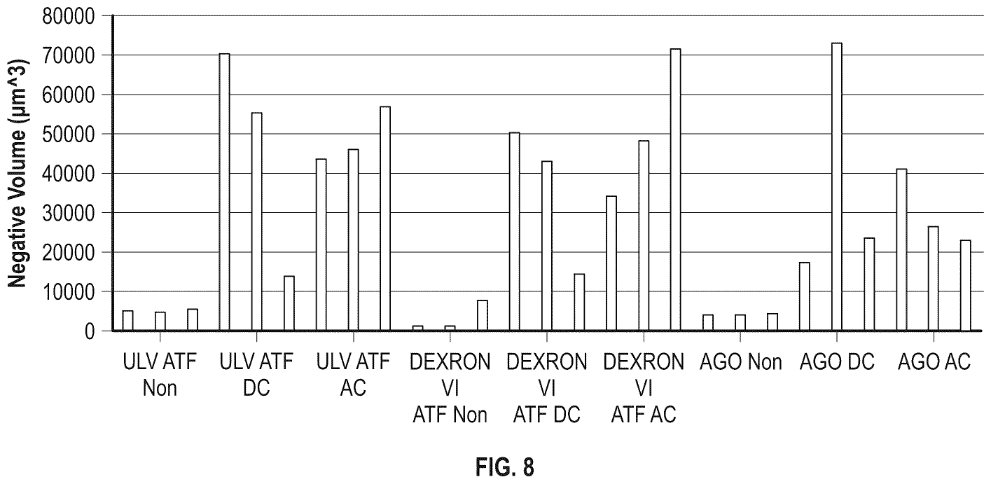

FIGS. 5, 6 and 7 show the traction coefficients for the three oils noted above and FIG. 8 shows end of test wear measurements. The nomenclature in the caption is as follows: oil-electrification-run number. For example, ULV ATF-Non-1 is ultralow viscosity automatic transmission fluid-nonelectrified-run 1. The left hand side of FIGS. 5-7 at zero rolling speed is boundary contact between the ball and disk and the right hand side of the graph is moving away from this condition towards mixed and then hydrodynamic. The higher the traction coefficient, the more contact there is between the ball and disk.

As can be observed, triplicate runs undertaken with no electrification are repeatable for all three oils. However, once either AC or DC current is applied, although they may start in similar places, they become different shortly into the test. Without being bound by the following, it is contemplated that with respect to tribological considerations, wear begins to immediately take place as testing starts and that such wear, due to the presence of electrification, changes the relative roughness of the running surfaces. Such roughness results in the same lubricant film thickness offering relatively less protection against separation of the parts and hence a relatively higher traction coefficient is observed in all the electrified tests. This is also shown in FIG. 8 by the relatively significant increase in volumetric wear at the end of the electrified tests over the non-electrified tests.

1. An electrically insulated ball on disk test unit comprising:
a. an electrically isolated rotating disk;
b. an electrically isolated rotating ball configured to be mechanically loaded against the electrically isolated rotating disk;
c. a negative electrode in electrical communication with the rotating ball and a positive electrode in electrical communication with the rotating disk, or a positive electrode in electrical communication with said ball and a negative electrode in communication with said disk;
d. wherein the negative and positive electrodes are configured to supply an electrical potential between the electrically isolated rotating ball and the electrically isolated rotating disk.

2. The electrically insulated ball on disk test unit of claim 1 further including a container configured to contain a lubricant or oil and said electrically isolated rotating disk.

3. The electrically insulated ball on disk test unit of claim 2 wherein said container includes one or a plurality of heaters configured to heat contained oil or lubricant.

4. The electrically insulated ball on disk test unit of claim 3 further including a thermocouple to measure temperature.

5. A method for evaluating the effects of electrical potential on one or more lubricants or oils in a test unit comprising:
supplying a test unit having an electrically isolated rotating disk, an electrically isolated rotating ball configured to be mechanically loaded against said electrically isolated rotating disk, a negative electrode in electrical communication with said ball and a positive electrode in electrical communication with said disk or a positive electrode in electrical communication with said ball and a negative electrode in communication with said disk, wherein said negative and positive electrodes are configured to supply an electrical potential between said electrically isolated rotating ball and said electrically isolated rotating disk;
introducing one or more lubricants or oils between said electrically isolated rotating ball and said electrically isolated disk;
rotating said ball relative to said disk; and
applying a mechanical load and an electrical potential between said electrically isolated ball and said electrically isolated disk.

6. The method of claim 5 further including heating said one or more lubricants or oils between said electrically isolated rotating ball and electrically isolated rotating disk.

7. The method of claim 6 further including heating said one or more lubricants or oils to a selected temperature.

8. The method of claim 5 further including measuring the friction between said electrically isolated ball and said electrically isolated disk.

9. The method of claim 5 further including measuring the wear track-volumetric wear between said electrically isolated ball and said electrically isolated disk.

10. The method of claim 5 wherein said lubricant or oil comprises automatic transmission fluid.

11. The method of claim 5 wherein said lubricant or oil comprises differential fluid.

12. A method for evaluating the effects of electrical potential on one or more lubricants or oils in a test unit comprising:
supplying a test unit having an electrically isolated rotating disk, an electrically isolated rotating ball configured to be mechanically loaded against said electrically isolated rotating disk, a negative electrode in electrical communication with said ball and a positive electrode in electrical communication with said disk or a positive electrode in electrical communication with said ball and a negative electrode in communication with said disk, wherein said negative and positive electrodes are configured to supply an electrical potential between said electrically isolated rotating ball and said electrically isolated rotating disk;
introducing one or more lubricants or oils between said electrically isolated rotating ball and said electrically isolated disk;
initially rotating said ball relative to said disk followed by applying a mechanical load and an electrical potential between said electrically isolated ball and said electrically isolated disk.

13. The method of claim 12 further including heating said one or more lubricants or oils between said electrically isolated rotating ball and electrically isolated rotating disk.

14. The method of claim 13 further including heating said one or more lubricants or oils to a selected temperature.

15. The method of claim 12 further including measuring the friction between said electrically isolated ball and said electrically isolated disk.

16. The method of claim 12 further including measuring the wear track-volumetric wear between said electrically isolated ball and said electrically isolated disk.

17. The method of claim 12 wherein said lubricant or oil comprises automatic transmission fluid.

18. The method of claim 12 wherein said lubricant or oil comprises differential fluid.
